(19) United States
(12) Patent Application Publication
(10) Pub. No.: US 2015/0224499 A1
(43) Pub. Date: Aug. 13, 2015

(54) Automated Microfluidic Sample Analyzer Platforms for Point of Care

(71) Applicant: SFC Fluidics, Inc., Fayetteville, AR (US)

(72) Inventors: Guochun Wang, Fayetteville, AR (US); Champak Das, Fayetteville, AR (US); Bradley Ledden, Fayetteville, AR (US); Qian Sun, Fayetteville, AR (US); Chien Nguyen, Fayetteville, AR (US); Sai Kumar, Johns Creek, GA (US)

(21) Appl. No.: 14/622,411

(22) Filed: Feb. 13, 2015

Related U.S. Application Data
(60) Provisional application No. 61/939,486, filed on Feb. 13, 2014.
(60) Provisional application No. 61/970,684, filed on Mar. 26, 2014.

(51) Int. Cl. B01L 3/00; G01N 33/53

(52) U.S. Cl. CPC B01L 3/502715; B01L 3/50273; G01N 33/5302; B01L 2300/0883; B01L 2300/0838; B01L 2300/0636; B01L 2300/0887; B01L 2300/046; B01L 2400/0415; B01L 2200/04; B01L 2200/10

(57) ABSTRACT

An automated assay platform for determining the presence and/or amount of analytes of interest in a sample at point of care integrates microfluidic enhanced assay sites, disposable cartridge designs, a sensitive low-volume detection module, together with selected pumping and valving modules, customized control board and user friendly graphical user interface (GUI). Comparing to traditional assay platform like 96-well ELISA, the platform is capable of reducing reagent consumption, increasing assay speed, and enhancing assay performance with a sample-in-answer-out automated process. This platform also features flexibility of adapting different assay schemes for different analytes.

### CROSS-REFERENCE TO RELATED APPLICATIONS

This application claims the benefit of U.S. provisional patent application No. 61/939,486, filed Feb. 13, 2014, for “Modular Microfluidic Assay Platform and Components”; and U.S. provisional patent application No. 61/970,684, filed Mar. 26, 2014, for “Microassay Devices for Measurement of Biomarkers.” Such applications are incorporated herein by reference in their entirety.

### STATEMENT REGARDING FEDERALLY SPONSORED RESEARCH OR DEVELOPMENT

This invention was made with government support under grant no. W81XWH-09-01-0523 awarded by the Congressionally Directed Medical Research Programs. The government has certain rights in the invention.

### BACKGROUND

This invention relates to assay components, assay devices and methods to improve assay outcomes, and more particularly to the integration of microfluidic technology and detection technology with established assay reagents for automated, fast sample analysis.

Immunoassay and enzymatic assay technologies for biomarkers are widely used for home, lab and clinical diagnosis. The traditional assay systems with these technologies include microplate based systems, tube/cuvette based systems and strip/lateral flow based systems. Microplate based systems are well established and broadly used in labs and some clinics. This platform still suffers from several drawbacks for point-of-care or home use.
1. Automation. To make the microplate assay automatic, a considerable amount of instrumentation, such as an automated dispenser, automated plate washer and automated plate changer must be paired with a special plate reader, a capability and resources most small labs and clinics will not have.
2. Portability. Most microplate based assay systems are bulky and not suitable for point-of-care applications. The required movement of optics and microplates would pose limitations on the ability to miniaturize the device.
3. Assay performance. For the most-often-used, 96-well assay platform, there are several assay incubation steps, which require up to eight hours to achieve satisfactory assay performance. The overall average assay time is often longer than four hours. Simply shortening the incubation time will result in much higher limits of detection, often above clinically relevant range of concentrations. More high density microplate platforms (384- and 1536-well) suffer from reproducibility issues and may require an additional robotic system for automated operations, which would significantly increase the instrumentation cost.

Tube/cuvette based systems are broadly used in centralized labs (such as Siemens ADVIA and IMMULITE system, and the Beckman Coulter ACCESS system). They are usually fast and sensitive; however they are not for the territory of point-of-care applications or research use because of the size, cost, special training requirements and availability of assays. Strip and lateral flow based systems are dominating certain biomarker diagnostic fields such as blood glucose and urine hCG level for their simple and fast assay process with very low cost. They are well suited for point of care and home use; however, very few low-abundant biomarkers have attained market success because of more stringent requirements in sensitivity, reliability and reagent requirements, especially for quantification. The most common technique for testing at the point of care (POC) is by use of the so called “Lateral Flow Assay” (LFA) technology. Examples of LFA technology are described in US20060051237A1, U.S. Pat. No. 7,491,551, WO2008122796A1, U.S. Pat. No. 5,710,005, all incorporated in their entirety by reference herein. Another technique for LFA is also described in WO2008049083A2, incorporated in its entirety by reference herein, which employs commonly available paper as a substrate and wherein the flow paths are defined by photolithographic patterning of non-permeable (aqueous) boundaries. Advances in LFA technology are disclosed in applications such as US20060292700A1, incorporated in its entirety by reference herein, wherein a diffusive pad is used to improve the uniformity of conjugation, thereby providing improvements in assay performance. Other disclosures such as WO9113998A1, WO03004160A1, US20060137434A1, all incorporated in their entirety by reference herein, have used the so-called “microfluidic” technology to develop more advanced LFA devices.

Tremendous efforts have been made to improve the microplate assay performance with a point-of-care platform, among which several instruments based on microfluidic technologies have been developed, such as i-STAT system (Abbott), TROVA system (Siloam Biosciences), and LABGEO analyzer (Samsung) based systems. Microfluidic based systems are ideally suited for assay based reactions as disclosed in U.S. Pat. No. 6,429,025, U.S. Pat. No. 6,620,625 and U.S. Pat. No. 6,881,312; all incorporated in their entirety by reference herein. The key advantages of microfluidic systems are the natural fit for automation, small sample requirement and high surface area to volume ratio that reduces the required assay time. However, it still remains as a challenge to easily adapt more analytes and perform multiple analytes assay simultaneously with a POC platform. It should also be noted that most label-free techniques do not at once meet the sensitivity, specificity, speed and reliability of detection of ultralow levels of many analytes. Immunoassays and enzymatic assays are often needed for specificity and sensitivity, and the protocol involves the use of multiple reagents and washing steps in a sequential programmatic manner—achieving this in the microfluidic format is formidably challenging. Furthermore, the assay results often lack accuracy without internal calibration since most reagents are vulnerable to environmental changes. Many instruments have tried to use a pre-stored calibration curve, but its practical value is limited especially when the assay conditions are changed. The expandability is another important aspect of such a tool, which means that it should be able to adapt new analyte tests or new assay methods easily by adding or exchanging new components. This is extremely helpful in developing new POC assays or performing POC service in resource-constrained environments. To the inventors' knowledge, there are no devices that cover every critical aspect described here. For example, Samsung's LABGEO analyzer, which is largely similar to the device described in U.S. Patent Application No. 20110269151, covers only several cardio vascular biomarkers without true on-site calibration. The assay format is restricted due to limitations of its centrifugal based fluidic control. Siloam Biosciences' TROVA system, which is based on US Patent Application No. 20120328488, is an open platform that can adapt many assay platforms, but its single channel pipetting fluidic delivery system may introduce cross-contaminations between reagents. Gravity and surface tension controlled flow are susceptible to sample quality and environment changes. Abbott's i-STAT system, which is related to many patents and patent applications (U.S. Pat. No. 8,017,382, U.S. Pat. No. 8,222,024, U.S. Pat. No. 8,642,322, U.S. Pat. No. 8,679,827, US20030170881, US20090065368, US20110290669, US20130224775), also focuses on several cardio vascular biomarkers besides simple ionic analytes. There is no on chip calibration for these immunoassay based tests so as to allow reliable and accurate quantitation, and they do not accommodate multiple immunoassays to be performed simultaneously.

### BRIEF SUMMARY

The present invention addresses limitations of the POC sample analyzer devices described above by introducing a modulated, fully integrated design. Components such as assay cartridges, pumps, valves, detectors, and sensors can be designed such that they are easily exchanged for different assay requirements in different implementations. Among these, several engineering designs and techniques are developed for quick fluidic connections between components, including quick-connect enabled connections, pierce-through self-sealing connections, and compressed O-ring connections. Integrated with specific assay cartridge designs and precise fluidic controls, a sample could in certain implementations be analyzed in less than one hour with built in on-site calibration. Multiple assay methods are easily adapted with different assay cartridges and protocols. Further extended designs are possible for simultaneous detection of multiple analytes. Therefore, the invention disclosed here is applicable not only to enable research lab and clinical diagnostics use, but also appropriate for specifically meeting point of care application requirements and emergency care in various implementations.

In various implementations, the invention provides a novel automated assay platform for determining the presence and/or amount of analytes of interest in a sample, comprised of uniquely designed component modules and related methods for point of care application. It is a versatile platform with potential of performing any immunoassays and enzymatic assays using a fast, sample-in-answer-out scheme. This platform uses modular designs to integrate disposable assay cartridge, sensitive onsite or offsite detections, precise flow control with pumping and valving system, an effectively error-proof feedback system and user-friendly graphical user interface (GUI). It is specifically designed and constructed to meet the point of care needs that traditional microplate-based systems, biochemical analyzer systems and strip-based systems do not address, because of lack of automation, large sample requirement, poor assay speed, large size of instrumentation and inadequate performance of the assays.

To perform a test in various implementations, the sample is introduced into the receptacle on the reagent compartment of the assay cartridge. After optional sample pretreatment, the assay cartridge is loaded into the system and the fluidic path is automatically established with the microfluidic system within the chassis with a convenient loading and unloading mechanism by means of quick connects, pierce-through connection, or compress-fitting. The user starts a predefined assay protocol with a user-friendly GUI and the test will automatically be run and the results will be reported once finished. The cartridges are disposable to minimize carryover. With the microfluidic design of the cartridge, the assay time and volume requirement are greatly reduced while keeping the assay performance. Real time calibration is built in with the cartridge so that variations from storage and reagent preparation can be minimized. Simultaneous detection of multiple analytes is also feasible with extended cartridge designs in certain implementations.

The detailed description and drawings provided herein will offer additional scope to certain implementations of the present invention. It should be understood that the described implementations are provided as examples only. Those skilled in the art will recognize that numerous variations and modifications of the described implementations are within the scope of the invention.

### BRIEF DESCRIPTION OF THE DRAWINGS

FIG. 1 shows an exploded view of an example system of the present invention wherein all components are integrated with one specific design (top and left sides of the enclosure 10 are not shown for clarity).

FIG. 2 shows an assay cartridge embodiment with microcapillary assay sites and a quick-connect enabled loading/unloading mechanism.

FIG. 3 shows an assay cartridge embodiment with inserted microcapillary tubing assay sites and a quick-connect enabled loading/unloading mechanism.

FIG. 4 shows an assay cartridge embodiment with spiral or serpentine assay sites with an optical quality sealer.

FIG. 5 shows assay cartridge embodiments with spiral or serpentine assay sites combined for multiple detectors or a single detector with a large aperture onsite detection.

FIG. 6 shows assay cartridge embodiments with spiral or serpentine assay sites sharing a common inlet or outlet port.

FIG. 7 shows assay cartridge embodiments with spiral or serpentine assay sites sharing a common inlet or outlet port and features for diffusion limiting.

FIG. 8 shows assay cartridge embodiments with spiral or serpentine assay sites sharing a common inlet or outlet port and introduced bypassing channel, together with selected optical quality sealer.

FIG. 9 shows assay cartridge embodiments with spiral or serpentine assay sites integrated with offsite on chip electrochemical detection.

FIG. 10 shows schemes of assay procedure for internal calibration.

FIG. 11 shows assay cartridge embodiments with reagent receptacles featuring quick connects for simple fluidic connections with the subsystem.

FIG. 12 shows assay cartridge embodiments with reagent receptacles featuring a self-sealing pierce-through mechanism for simple fluidic connections with the subsystem.

FIG. 13 shows assay cartridge embodiments with integrated reagent and assay compartments.

FIG. 14 shows assay cartridge embodiments with a separate fluidic connection chip.

FIG. 15 shows the aperture operation for large aperture detectors.

FIG. 16 shows a chip loading mechanism with compressed O-ring seal.

FIG. 17 shows an on chip sample preparation with filtration and centrifugation.

FIG. 18 shows possible assay methods with certain implementations of the invention.

FIG. 19 is a set of bar graphs showing decreased performance of 96-well plate assay with accelerated steps.

FIG. 20 is a graph showing system performance with a dye test.

FIG. 21 is a set of graphs showing system performance with offsite detection.

FIG. 22 is a graph showing system performance with an IL6 assay.

FIG. 23 is a set of graphs showing system performance with a T3-T4 competitive assay.

FIG. 24 shows system performance with simultaneous detection of IL6 and GFAP assay.

### DETAILED DESCRIPTION

In various implementations as described herein, the invention features a modular, open design architecture for automated analyte analysis at point of care. A more complete understanding of the apparatus, components and operations can be obtained by reference to the accompanying drawings, as follows.

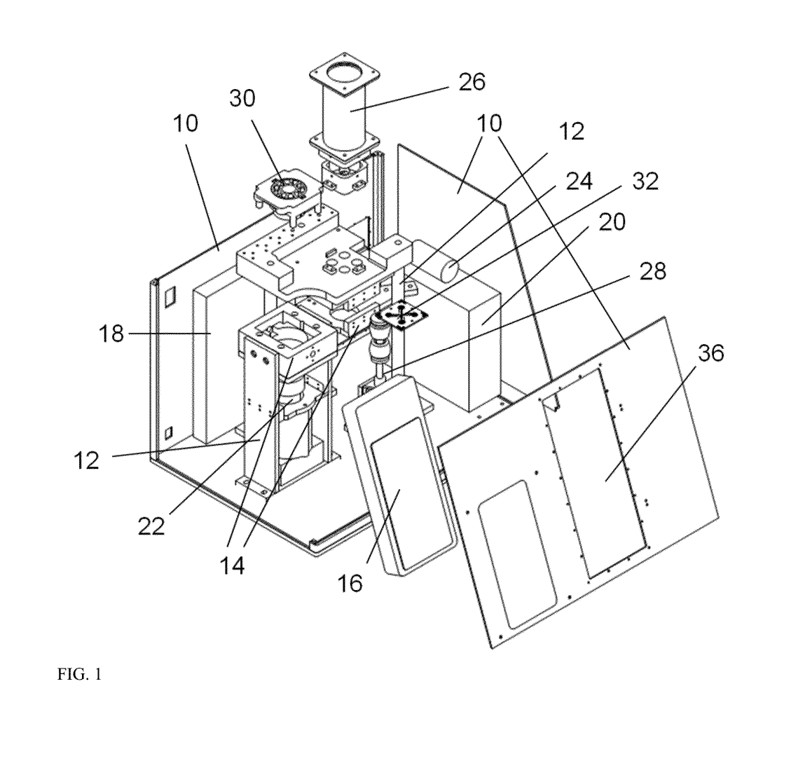

FIG. 1 is an overview of an exemplary device. This device has been tested for many protein analytes, especially those related to traumatic brain injury (TBI). It is a fully automated, modular microfluidic platform capable of rapid ultrasensitive analyte detection. It capitalizes on the advantages of using on-chip reaction and detection with sample requirement less than 60 μL, and controlled flow for precisely programmed execution of multistep assay protocol. Other features include:
1. Low detection limits: demonstrated at 10 μg/mL for IL6 and at 50 μg/mL for GFAP with serum samples.
2. Fast, quantitative results: the disclosed system could simultaneously detect up to four samples with total time less than 1 hour (depending on the specific analyte) for the automated sequence from sample collection to assay results. The use of on-chip calibration enables reliable quantitation due to obviation of chip-to-chip variation.
3. Disposable components: Both reagent cartridges and assay chips are single-use disposable plastic parts, intended to minimize potential cross contamination.
4. Customizable platform: because of the open, modular architecture, the adaptation of new analyte assay is straightforward.
5. On-chip detection: Real time on-chip detection not only increases the detection sensitivity, but also speeds up the assay. It can supply both kinetics and end point information depending on assay requirements.
6. Portable size for POC use: The targeted size of the system is about 9″×11″×15″ with integrated nurse-friendly touch screen.

TABLE 1
Specifications of exemplary system
in FIG. 1

| | |
|---|---|
| Assay time | 30-60 min |
| Sample requirement | 60 μL |
| Samples used | Serum/plasma/CSF |
| Size of assay chip | 1.5″ × 1.5″ |
| Flow rate | <45 μL/min |
| Pressure | 0-14 psi |
| Detector | PMT |
| Dimension | 9″ × 11″ × 15″ |
| Limit of Detection | 10 pg/mL |

The specifications of the exemplary device are shown in Table 1 and the detailed configuration is shown in FIG. 1. This example system is composed of several fixtures and replaceable modules to easily meet different requirements of analyte analysis. The fixtures include the enclosure 10, chassis 12, assay chip loading station 14 (assay chip tray 158 and linear actuator 156), touchscreen PC 16 and the multifunction control board 18. The multifunction control board is designed for adopting various modules that could be used in the system, including pumps 20, valves or manifold 22, sensors 24, detectors 26 (both optical and electrochemical), and actuators 28. Some other useful modules like sample preparation, reagent mixing and light source modules could be also implemented. These replaceable modules make the disclosed system fully open and ready for various analyte analysis. The assay cartridge is fully disposable and a separate design of reagent 30 and assay 32 compartments is shown in FIG. 1. These two compartments could be combined to a single embodiment 34 as discussed later. All the components are packed in a light-tight enclosure 10 with at least one opening door 36, which is used for loading and unloading assay cartridge 34. More openings are optional, especially for two individual compartment assay cartridge design and for easy maintenance purposes. By using different sets of assay cartridge and a modified assay protocol, different target analytes could be measured in the same way with the same device. For the overall assay process, preloaded assay reagents and samples are loaded from reagents compartments 30 through microfluidic subsystem over to the assay sites 32. Each step of reagent loading, incubation and removal are precisely controlled by predefined assay programs and through pump 20 and valve 22 systems. The valve options include multichannel valves and manifolds with fluidic control of a plurality of microfluidic connections. The final detection, data analysis and report are processed automatically with the embedded PC system 16. Several sensors 24 are also integrated for real time assay monitoring and troubleshooting. The sensors include but are not limited to flow sensor, pressure sensor and temperature sensor. An inline flow sensor is very useful to provide real time flow information during the assay and could detect variations caused by clogging, bubbles and valve operations. A pressure sensor that connected to the fluidic system through a manifold could also provide real time flow information to prevent clogging and potential leakage during the assay. A temperature sensor could monitor the local environment for the assay. Once paired with a heating/cooling module, the temperature sensor could help maintain the system operated at the optimal temperature range for assay reactions. Many biomarkers are easily adapted to the analyzer because of its open configurations and some of the tested TBI biomarkers are shown in Table 2. More details of certain biomarkers are described later.

TABLE 2
TBI biomarkers tested with the example device shown in FIG. 1.

| Biomarker | LOD | | Dynamic range | Spiked recovery |
|---|---|---|---|---|
| IL6 | 10 | pg/mL | 3 Logs | Within 20% |
| GFAP-BDP | 50 | pg/mL | | Within 25% |
| BDNF | 50 | pg/mL | | NA |
| S100b | 50-100 | pg/mL | | NA |
| UCHL1 | ~0.1 | ng/mL | | NA |

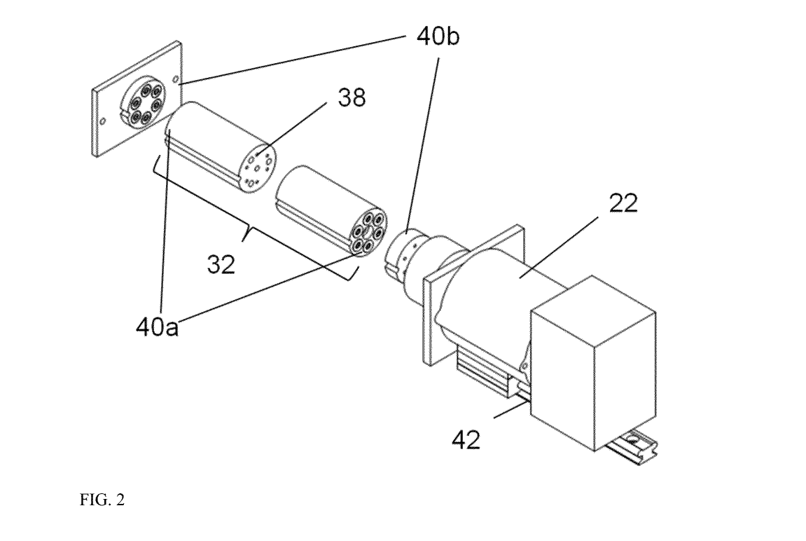

A key concept to improve the assay performance with automation herein is the combination of microfluidics with assays. Micro features enable extremely large surface area to volume ratio, so that for diffusion limited assays (including most enzyme-linked immunosorbent assay (ELISA) assays since the kinetics of antibody/antigen reaction is much faster than the diffusion process), the theoretical required assay time and assay volume is greatly reduced (the actual number varies based on specific designs). The automation feature is achieved from the inherent fluidic mode with the interface to precise fluidic control. FIG. 2 shows an example of assay cartridge design that features microfluidic assay sites 38, quick-connect enabled fluidic connections 40a 40b and convenient slide-in loading mechanism 42. Quick-connect enabled fluidic connectors are described, for example, in U.S. Pat. No. 8,337,783 and U.S. patent application Ser. No. 13/417,538, each of which is incorporated by reference herein. The embodiment of the cartridge 32 has six microcolumns 38 across the body with the same diameter (<1 mm). The surface of the microcolumns is modified with analyte assay specific receptors with proper immobilization methods (either adsorption, entrapment, or chemical modification based on surface material, receptors, and coating protocols). All the assay steps are processed on microcolumns and the final signals are measured with a downstream detector. To achieve fast but reliable fluidic connections between the cartridge and the fluidic system in the device, quick connects 40a 40b are used, paired with a sliding station 42. Quick connects 40a are integrated at both ends of the cartridge and they could self-align and connect to the complementary adapter sides 40b in the system. One adapter side is fixed while another side is sitting on a moving station 42 so that the engagement and disengagement of the cartridge with the system is freely done and guarantees the full connection force from quick connects. Quick connectors 40a 40b of ¼″ size are demonstrated in FIG. 2, but the preferred size could vary depending on number of assay sites and force requirement for reliable connection. The design in FIG. 2 has to overcome several engineering challenges. The surface area and volume are critical to the assay performance and a reliable manufacturing method is relatively hard to achieve.

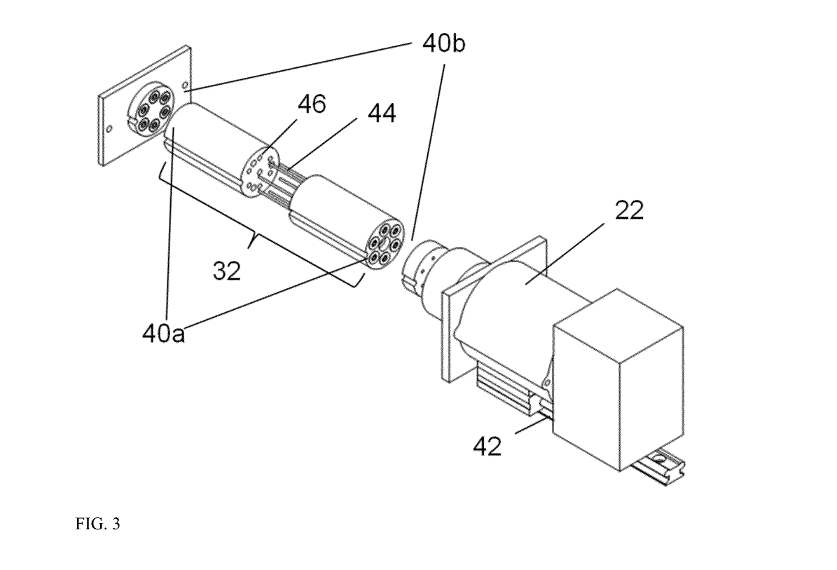

As an alternative design to minimize the potential engineering challenges for the microcolumn features, the whole embodiment 32 in an alternative implementation could be a housing design for embedded capillary columns 44 as shown in FIG. 3. Instead of making long microcolumns directly, precoated capillary columns 44 are assembled through much larger apertures 46 on the embodiment 32. Only the two ends of the embodiment are critical for fluidic connections, which are already handled with quick-connect designs 40a. Another advantage of this design is that the capillary tubings are available at various sizes with various materials, thus more coating options are feasible for different analytes. Materials such as Polytetrafluoroethylene (PTFE), Polycarbonate (PC), polystyrene (PS), Cyclic olefin copolymer (COO), Poly(methyl methacrylate) (PMMA) and fused silica with ID sizes ranging from 200 μm to 750 μm have been tested successfully with the analyzer. Furthermore, since the surface property of these commercial quality capillary tubings is known, a prescreen process would ensure better microcolumn reliability. It also brings convenience for reliable receptor coating since a batch of long capillary tubings could be coated with the same solution in the same way before cutting into microcolumn sizes. This is a paramount step to improve the overall system performance.

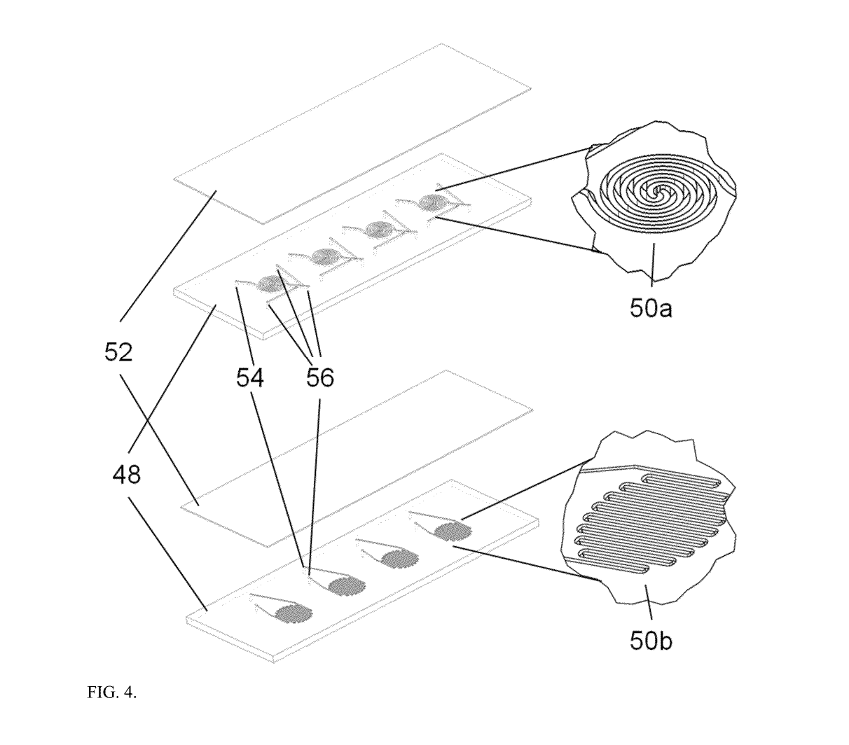

With the microcolumn design, an example device demonstrated very good performance with model assays as described later, however, faster assays with better performance could not be achieved due to the physical limitations of microcolumns (not compact and no onsite detection). A chip format was therefore chosen. FIG. 4 shows two design examples of on-chip assay sites that could be used in implementations of the system. Both designs include an embodiment 48 (1-3 mm thickness) with microfluidic channel enabled assay sites 50a 50b and a sealer 52. Both spiral (50a) or serpentine (50b) designs are viable with spiral design providing better flow profile because of less sharp turns on the geometry. The microfluidic features are densely packed for onsite reaction and detection. The channel width could vary from 100 μm to 500 μm. The channel to channel gap could vary from 200 μm to 400 μm and the depth of channel could vary from 50 μm to 300 μm. Smaller overall features may be chosen, but could bring engineering challenges and deteriorate reliability. Each assay site has one outlet 54, but could have multiple inlets 56 for loading of different reagents as shown in FIG. 4. These ports are connected from the back of the embodiment with the sub-fluidic system in the device.

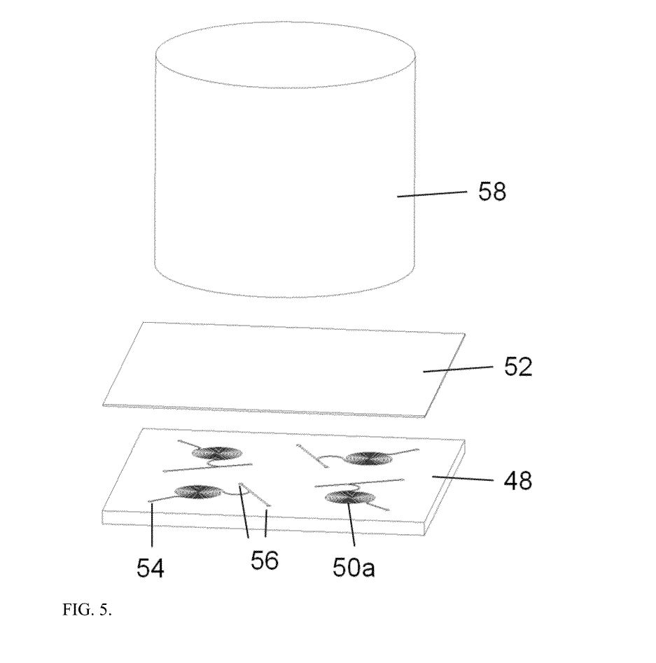

Onsite detection is another main advantage with the designs in FIG. 4, as shown in FIG. 5. An optical quality sealer 52 is used to seal the embodiment 48 and optical detectors 58 could align with each assay sites for detections based on either fluorescence or luminescence. Multiple detectors for individual assay sites (such as photodiode array) are an option to minimize the moving parts, but the variations between different detectors could adversely affect the assay results. Instead, single large aperture detector 58 (PMT or camera) with properly designed assay chips could gain the best reliability as an example shown in FIG. 5. All the spirals 50a are organized within a 1.5″×1.5″ chip 48 so that a large aperture detector 58 (e.g. Hamamatsu PMT H11870-100, CCD camera H10990-904, and Andor CCD camera Luca) could be directly mounted on top of it with an extension tube without additional optics. Both cartridges 48 and detectors 58 are not required to move during the detection and could potentially get the best signal directly from the assay sites.

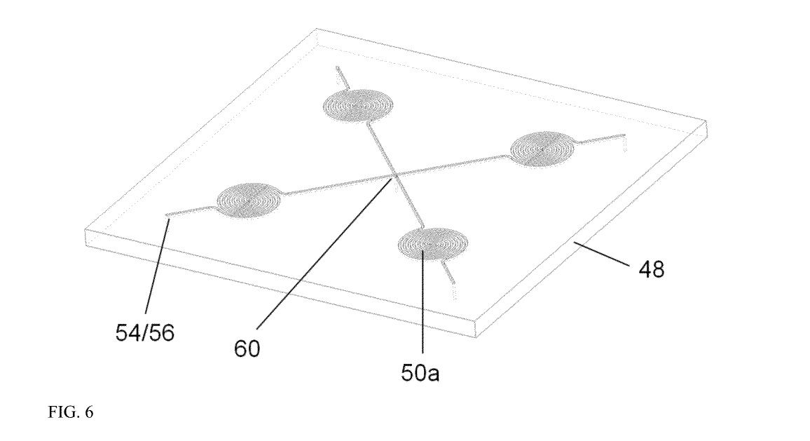

Assay chips 48 have ports 54 and 56 open at the backside for fluidic connections. The more open ports, the more complicated a subsequent fluidic connection will be. Thus individual addressable assay sites are expected to have engineering challenges later on for assay automation. Instead, either all inlets or outlets could be combined to one single port 60 to greatly reduce the complexity while keeping a similar or better assay performance (FIG. 6). When used as a common outlet, the potential crosstalk between assay sites is minimal because all diffusions around the common port are easily washed out before entering the assay sites. When used as a common inlet, the requirement on reagents will be minimal since they are not required to route through an external fluidic embodiment (valve, manifold, etc.). The actual optimal configurations depend on targeted assay requirements.

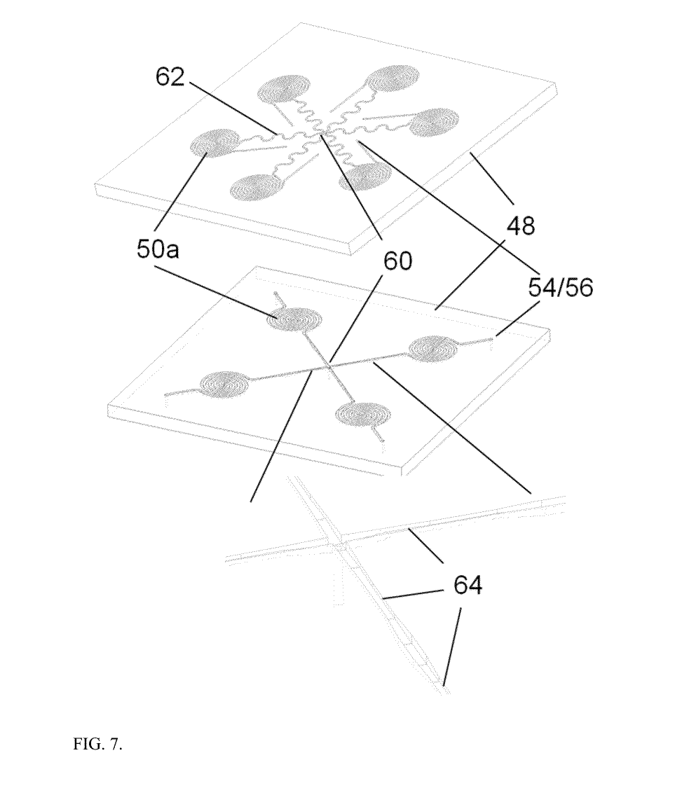

There are several ways to minimize the potential assay variations due to diffusion from the common port 60 and two of them are shown in FIG. 7. One method is to elongate the connection channel from the common port 60 to the assay site 50a by introducing another serpentine feature (62). The longer the serpentine channel, the less effect of potential contamination to the assay site 50a from the common port 60, however, it takes more volume and space on the chip. Thus the configurations are balanced based on the protocol and reagents used. A second approach is to set several fluidic restriction sites 64 on the connection channel (FIG. 7 bottom with the close-up), wherein narrower and shallower sections 64 could slow down the overall diffusion process while keeping the transition volume even less, with little effect to engineering challenges. The third option for diffusion limiting is to control the surface properties (such as hydrophobicity), however, this might be of limited use because of complexity of reagents used in an assay. Furthermore, FIG. 7 (top) also shows an example of packing more assay sites 50a into the same size assay cartridge, which could be helpful for more simultaneous assays. Actually, there is no theoretical limit to the assay sites per assay cartridge in alternative implementations.

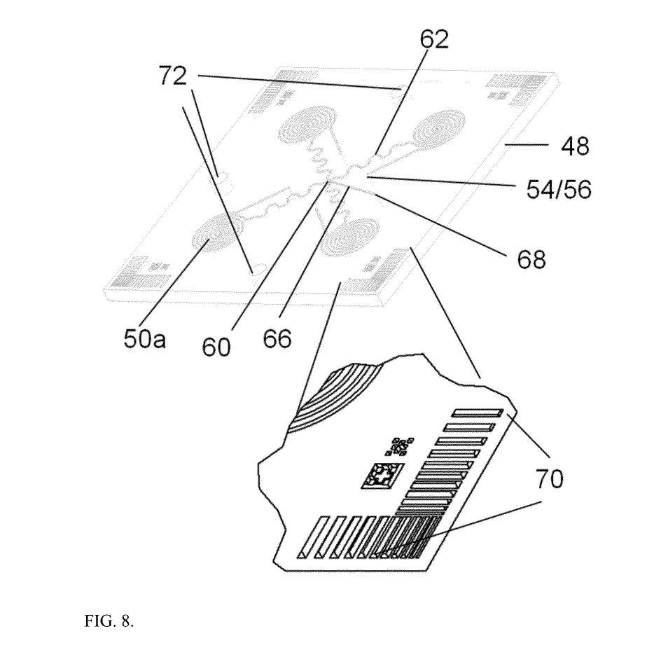

Since most assays involve multiple reagents, the efficiency of previous solution removal greatly affects the performance of later reagents. Generally, bypassing tubing will be introduced to clean out the solutions in the fluidic subsystem with new solutions and not disturb the assay sites (for previously-described implementations shown in FIGS. 2, 3, 6 and 7) before passing through the assay sites 50a and 50b. An alternative approach is to include the bypassing feature on the chip itself, as shown in FIG. 8. The bypassing channel 66 is a short channel that directly connects the common inlet (outlet) 60 and outlet (inlet) 68 so that a new solution could prime the system without affecting the assay sites 50a. It is usually designed to be short in length for minimum transition volumes. FIG. 8 shows a fully functional test example chip 48 with four spiral assay sites 50a. The chip material could be PC, PMMA, PS, COC or even glass. Opaque material is preferred for on-chip optical detections. The sample chip is 1.5″×1.5″ with 1/16″-⅛″ thickness to keep certain stiffness and prevent from deformation after loading. The channels are 200 μm wide with 140 μm depth. Channel to channel wall is 300 μm thick. Positive control, negative control and a duplicate of samples could be tested simultaneously. Each spiral takes about 3 μL volume and the sample requirement is less than 60 μL. Besides the spiral structure 50a, some benchmarks 70 are located around four corners for fabrication quality controls. Small wells with different depth and width wells are fabricated together with the spirals to make chip quality control much easier (such as channel depth and wall thickness variations could be checked with these benchmark wells). Three align holes 72 could be used for precise chip mounting, besides an edge reference that could also be used for precise chip alignment with the fluidic subsystem.

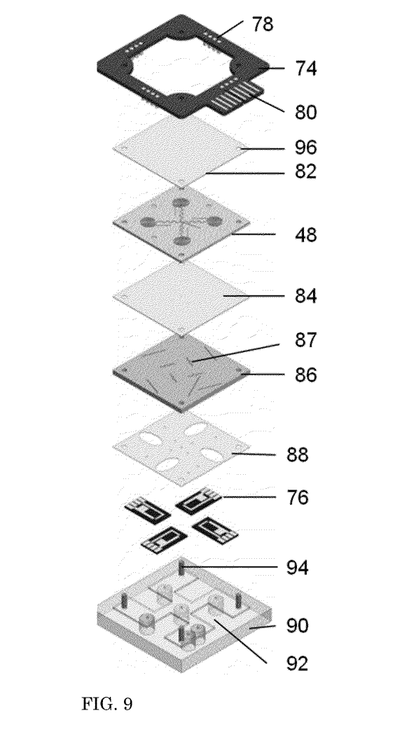

In addition to onsite optical detection with the device, the analyzer can also be adapted to measure assay results electrochemically with offsite electrochemical detectors. Electrochemical (EC) detection as performed here requires the detectable species to be transported (by flow) to the electrochemical sensor. This complication is due to the fact that electrochemical measurements are surface sensitive making it difficult to perform the full assay on the sensor surface. For this reason an example cartridge is shown in FIG. 9 that permits the assay chip and sensing electrodes to be packaged together. The cartridge is designed in a “layer cake” format with individual layers performing separate functions. As shown in FIG. 9, the top layer 74 is the electrical interface layer which contacts the sensor chip 76 through four spring contacts 78 and permits a card edge connector to make electrical contact 80 with measurement electronics. Layer 82 in FIG. 9 is the plate sealer tape that seals the assay chip 48. Layer 84 is a double-sided adhesive layer that serves to connect the assay chip 48 with layer 86 which is the fluid interface layer. Layer 86 serves to carry the detection solution from the assay spiral 50a in the layer above to the EC detector chip 76 in the layer below. The electrochemical assay cartridge is accessed fluidically through a valve 22 beneath the cartridge. In this case a ten-port selector valve 22 is used to address all the fluid paths of the cartridge. The solution is introduced through the central port 60 and then flows to the respective spiral 50a drawn by suction generated by a syringe pump 20. The fluid then exits the assay chip 48 and passes straight through the fluid interconnect layer 86 and out to the valve 22 for most of the assay steps. During detection, however, the valve 22 switches to pull the solution down a serpentine channel 87 and onto the EC detector chip 76. After passing over the detector chip 76 the solution again passes though the valve 22 and out to waste. Layer 88 in FIG. 9 is another piece of double-sided adhesive that not only adheres the fluid interconnect layer 86 to the detection chips 76 but serves as a gasket to form a flow chamber on the detection chip 76 as well. The geometry of this gasket is important to ensure proper flow of reporter molecules from the assay spiral 50a across the entirety of the sensing region of the detector chip without permitting the trapping of bubbles. Finally the gasket layer 88 also serves to adhere the fluid interconnect layer to the bottom layer 90. The bottom layer has recesses 92 that position the detection chips 76 both laterally as well as height-wise so they are able to make proper contact with the adhesive gasket layer 88. The bottom layer also has four dowel pins 94 that serve to position the layers above. Each layer has a set of four guide holes 96 that align the individual layers. This alignment procedure is enough to enable all the fluid vias to align for the different layers. The fluid vias between the different layers are 500 μm diameter, double the size of the channel widths in the layers themselves to facilitate easier alignment. After assembly the cartridge is placed in a Carver press under 500-1000 psi of pressure to ensure the layers are laminated together properly.

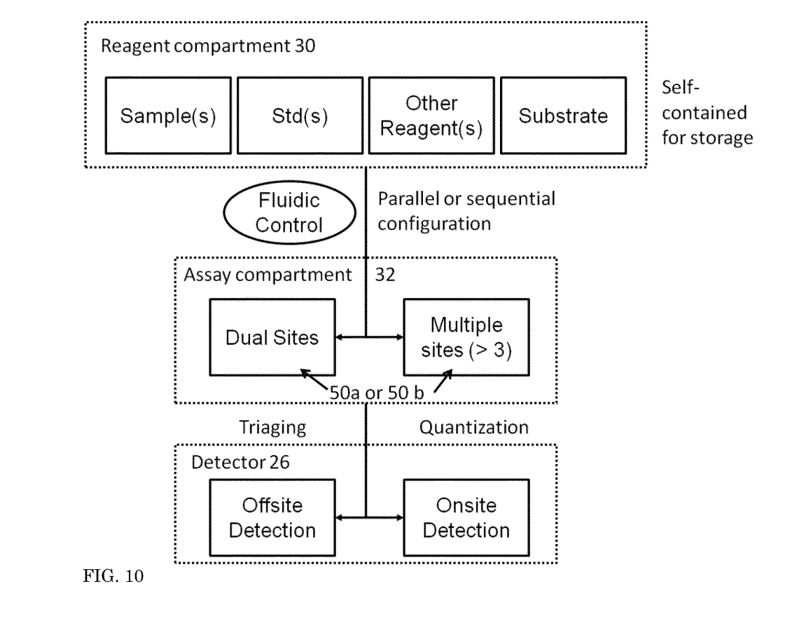

No matter what detection method is used (onsite or offsite), onsite real-time calibration is another feature that enables reliable assays with the device. Similar to traditional 96-well plate assay, wherein a calibration curve is always prepared together with sample measurement to eliminate uncertainties from reagent degradation, plate differences, concentration variations and environment changes, internal standards are included in this system as shown in FIG. 10. A functional reagent compartment 30 includes sample receptacle, standard solution(s), together with other reagents and substrate. They are designed to be physically separated from each other to avoid contaminations during storage. All reagent receptacles are sealed with water impermeable sealers for longer time storage. During a test, all the samples and standard solutions are loaded to different assay sites of the assay compartment 32, while all other reagents are shared. Reactions on each assay sites 50a 50b could be either parallel or sequential. Parallel reaction means all reagents pass through all assay sites at the same time, which usually requires individual fluidic control for each assay site. On the other hand, reagents pass though assay sites in an orderly sequential mode, which reduces the complexity of the system design. Both options are viable depending on assays. On-chip calibration requirement is also depending on the assay requirements. For triaging assay tests, a cut-off value concentration of standard analyte is enough. It can be used for direct comparison with the measured value and the qualitative test result is a simple yes or no. For more precise quantifications, more assay sites are required to have a full calibration curve on site, similar to that from 96-well plate assay platform. Generally more calibration sites should increase the reliability. A quick test comparing three calibration points and four calibration points with IL6 assay on the system showed more than 10% signal enhancement. The overall number of calibrators has to be determined based on assay performance and system complexity.

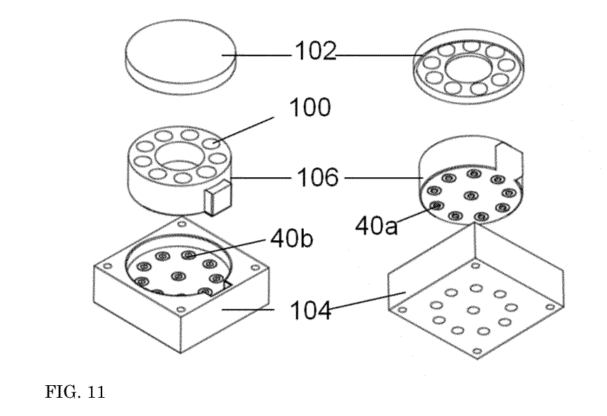

A reagent compartment 30 could be independent from the assay compartment 32 or combined together. Since it contains multiple solutions and interfaces the sample, the loading, and the unloading mechanism is more complicated. There are four main challenges for a user-friendly disposable reagent compartment. First, the reagent should be stored for a long time without leakage/evaporation. Second, loading and unloading the cartridge to the system should be simple. Third, an automatic fluidic connection should be set once loaded. Fourth, it should not have any leakage after unloading of the cartridge. To address all these challenges, two innovative designs are introduced in various implementations of the system. One example design that features quick-connect connectors is shown in FIG. 11. The reagent compartment 30 has ten wells 100 to accommodate all the solutions including sample, standards, secondary antibodies, detect reagents, substrate, and washing solution. The bottom of the cartridge features individual quick connects 40a. Bottom side is sealed with water impermeable sealer (not shown in FIG. 11) while top side is sealed with a cover 102. When loading, the bottom seal is removed and solutions will remain in the cartridge due to surface tension effect. The compartment 30 could be dropped into the mating adapter 104 on the device. The fluidic connections are automatically established because of the self-alignment feature from the quick connects 40a and 40b. After removing the top cover 102, the solutions are ready to be pulled into the device. Once the assay is finished, the top cover 102 can be replaced and the cartridge 30 could be safely removed.

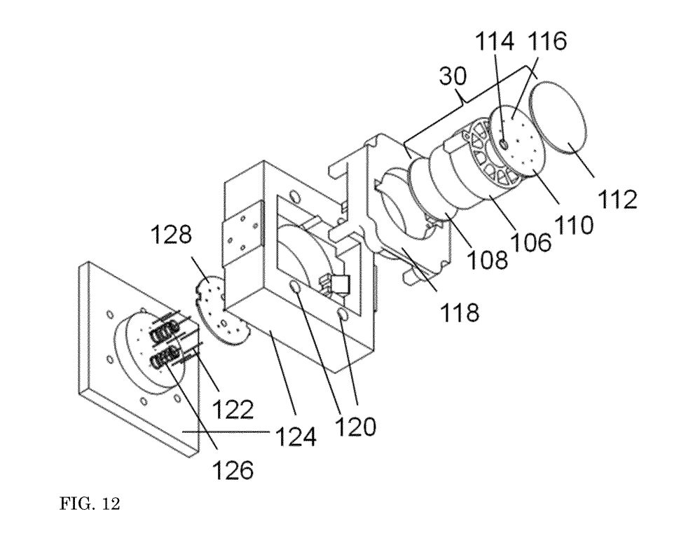

In this quick-connect based design, there is still a chance of solution leakage during cartridge loading and unloading because of risks from capillary force holding solutions. Another design example shown in FIG. 12 features a pierce-through mechanism. In this design, the fully assembled compartment 30 contains four parts: body 106, bottom sealer 108, top cover 110 and top sealer 112. The bottom sealer 108 is a thick elastic membrane. The top cover 110 is rigid with one large opening 114 for the sample loading and ventilation holes 116 for other wells. Depending on the stability of the substrate, the substrate well might be an empty well or a prefilled one. The prefilled cartridge 106 will then be sealed with low water permeability sealer 112. The fully assembled cartridge 30 could be stored properly for future use. To load the assay cartridge 30, the top sealer 112 would be peeled off during the test to reveal the ventilation holes 116 and sample loading well 114. After loading sample to the sample well 114 with a pipette or similar mechanism, the reagent cartridge 30 would be loaded to the system through a matching adapter 118 with integrated orientation feature. Four magnets 120 are used to keep the cartridge 30 down and secured in place. Unloading of the cartridge is also simplified with this magnet design. There are many needles 122 located at the bottom of the adapter 124 and each of them is aligned with one reagent receptacle. Fluidic connection is established with the needle connectors 122 piercing through the elastomeric seal 108 at the bottom of the cartridge 30. The material of the bottom sealer 108 is selected for self-sealing of holes after punctured by needles, which is not only important to prevent solutions from leaking during test, but also keep the solutions in place after cartridge removal. The needles 122 are normally protected under a spring 126 loaded guard plate 128 to prevent accidents. Needles 122 are only exposed once the reagent cartridge 30 is loaded to press the guard plate 128 down. After each assay, the operator just needs to seal the cartridge with the original sealer 112 and take it out. The cartridge 30 is ready for disposal without any contamination risks.

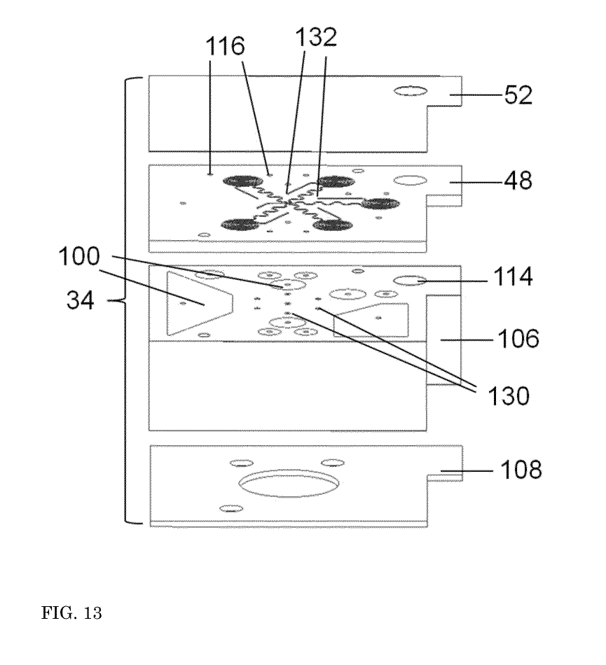

As an illustration of combining reagent and assay compartments together, one design according to an implementation of the invention is shown in FIG. 13. Combining the two parts 30 and 32 into one cartridge 34 has several benefits. First, user errors due to mismatched (incompatible) components will be eliminated. Secondly, the error due to misalignment is reduced since it will be virtually impossible to improperly insert the new cartridge with integrated alignment feature. An integrated assay cartridge will also permit better quality control since the reagents and receptor coated assay sites are analyte-specific and will be correlated during manufacture, ensuring lot-to-lot compatibility. This permits the use of a single expiration date for one disposable module. This is important when the system is used to perform multiple analyte measurements.

In addition to reducing user error, an integrated assay unit permits some simplifications in the device hardware as well. The simplest of these improvements is the use of fewer openings in the device, thus simplifying the light-tight chassis manufacturing. Another improvement enabled by integration is the reduction of transit (dead) volume which translates to shorter assay time and reduced reagent consumption.

In the example of FIG. 13, both reagent 106 and assay 48 components are manufactured by injection molding. This permits small feature sizes and tight tolerances to be preserved on the assay channel molding using the precision mastering. After molding, the assay component 48 has ports 132 drilled and trimmed to size. Then it is coated with receptors and blocked with blocking reagents after sealing with an optical quality sealer 52 on top. The final step is to dry the assay chip 48 before integration with reagent component 106. The reagent component 106, on the other hand, has comparatively less stringent molding tolerances. The bottom of the reagent reservoir is sealed with an elastomeric seal 108 that permits access to the reagents by puncturing with needles 122. There are many vias 130 drilled through the body that match ports 132 from the assay chip 48 to lead solutions. The reagent wells 100 are filled with individual reagents and the assay chip 48 is used to bond on top of the reservoir block 106 by means of the double-sided tape applied to the bottom side of the assay chip (not shown in FIG. 13). The finished cartridges 34 are barcoded and sealed for storage. The use of the integrated assay cartridge is as simple as drop-in the reagent compartment 30 shown in FIG. 12.

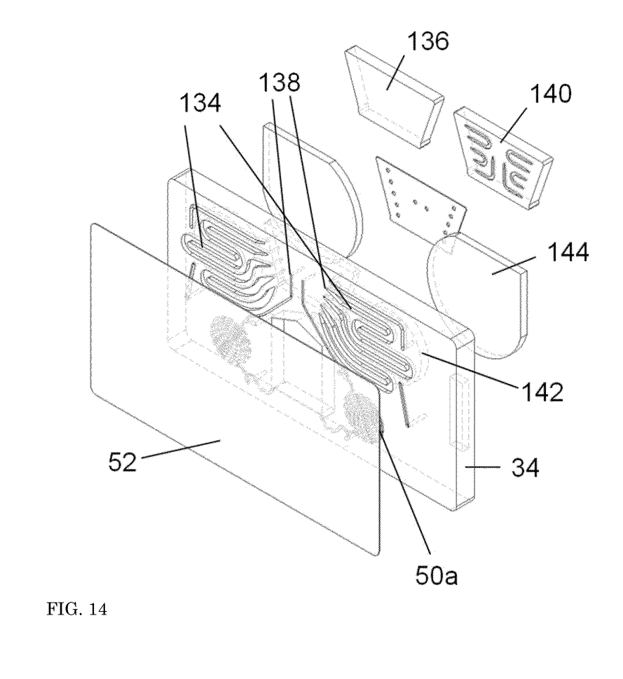

A further simplified design of combination is shown in FIG. 14 for the same chip configurations. The idea is to have all the reagents 134 required for assay (except samples) stored on chip while separated with a septum 136 to cover all the ports 138. A separate connection channel chip 140 is used to replace the septum 136 and the fluidic connections are automatically established from the reservoirs 134 to the assay sites 50a once assembled. The substrate solution could be stored under a separate reservoir 142 with elastomer membrane 144, which could supply pressure driven flow for all reagents once activated with an actuator. Assay sites 50a are spiral configurations similar to other designs as described herein for onsite detection. The overall volume and assay time in this design could be greatly reduced due to extremely small transition volumes. Besides using an actuator, the pumping mechanism could be traditional pumps, or electrochemical pumps for their extreme smooth flow at a flow rate less than 100 μL/min. Suitable electrochemical pumps include those described in U.S. Pat. Nos. 7,718,047 and 8,187,441, each of which is incorporated by reference herein. The complexity of the assay chips will rely on the assay protocol. In the most complicated situation as a full-blown ELISA assay, there will be a total of five solutions and seven steps for one sample measurement. On the contrary, there will be as few as two solutions and two steps with premixing strategy for one sample measurement (FIG. 14). The chip design and the assay performance should be balanced.

Ideally, one measurement of the sample would be sufficient to give positive or negative answers by comparing to the predefined cutoff value. However, without an internal standard, it would be difficult to correlate the measured signal value with the actual biomarker concentration. Thus a two-spiral chip design is more practical for actual use. As shown in FIG. 14, one of the two assay sites 50a will be used for sample measurement, while the other site will introduce the biomarker at the cutoff value. By comparing the sample value to that of the “spiked” standard solution, a triaging decision could be quickly made.

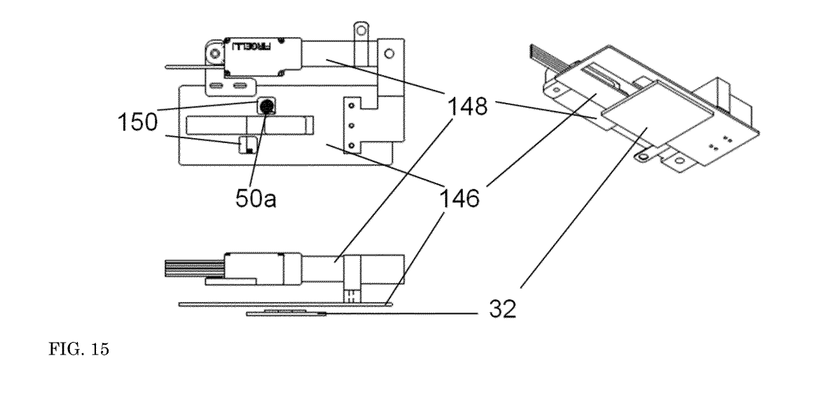

The design of using a single large-aperture detector for best reliability is shown previously in FIG. 5. It is possible for a large-aperture camera to define different signals from different assay sites simultaneously. In this case, a complicated image processing method has to be defined in the control software. An alternative way is to introduce a shuttering mechanism, as an example shown in FIG. 15. A special designed shutter 146 driven by an actuator 148 is used to expose one assay site 50a at a time. It could either be linear actuator 148 as shown, or a rotary shutter as most filter changers do. In FIG. 15, the linear shutter 146 is placed close to the chip. One and only one assay site 50a is exposed completely once aligned with a predefined aperture 150 on the shutter. The lights from the neighbor sites are minimized with such a close placement and black matte surface around. The measured signal can be directly used for kinetics or end-point analysis without complicated data processing.

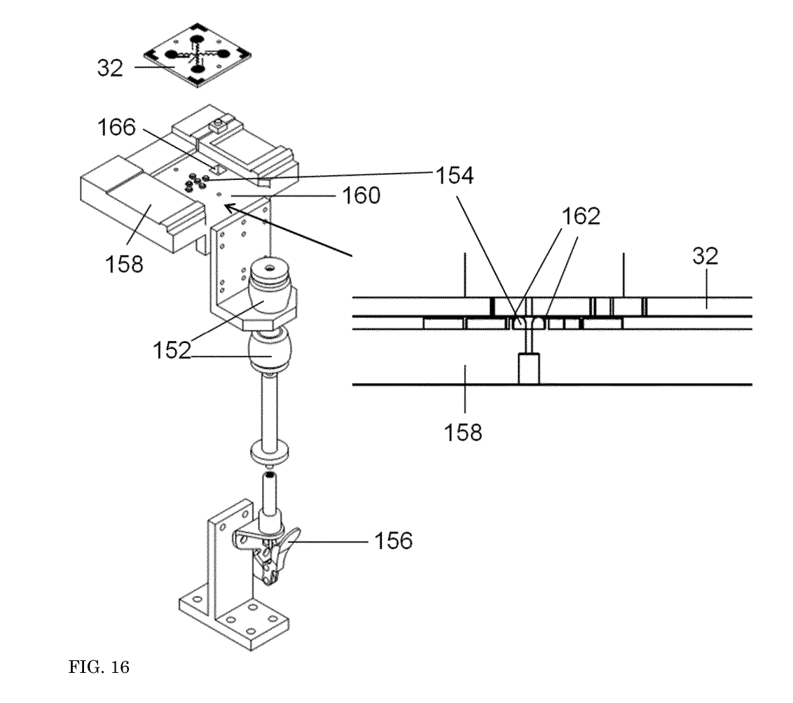

A specially designed assay chip loading mechanism is developed as shown in FIG. 16 since quick-connect design does not fit because of the geometry constraints and the challenges to seal multiple sites on the same plane in some circumstances. Instead, a spring-loaded actuator 152 paired with compressed O-ring seals for O-ring sealed ports 154 is employed. The key features are the linear actuator 156 and the redesigned assay chip tray 158. The assay chip tray 158 has a chip insertion slot 160 and three edges are designed to precisely define the position of the chip for fluid connections. In the center of the tray are six raised O-ring sealed ports 154, which match the ports on the inserted chip 32. The raised bed 162 feature ensures proper contact between the chip 48 and the O-ring 154, but not other parts of the assay chip tray 158, which concentrates the force over the O-rings for better sealing. The linear actuator 156 will raise the assay chip tray 158 together with the chip 32 and against the top optical assembly. It is spring loaded to tolerate certain variations from chip thickness. With properly adjusted actuation force, which could be fine adjusted with an integrated pressure sensor 166, the fluidic connection between the chip 32 and the valves 22 downstream is automatically set without leakage or clogging and the assay could be started. Just toggling the linear actuator 156 to lower the chip tray 158 and the chip 32 could be removed from the front. Further design could introduce a motorized actuator controlled by the central board 18 with the feedback from the pressure sensor 166 for automatic pressure control.

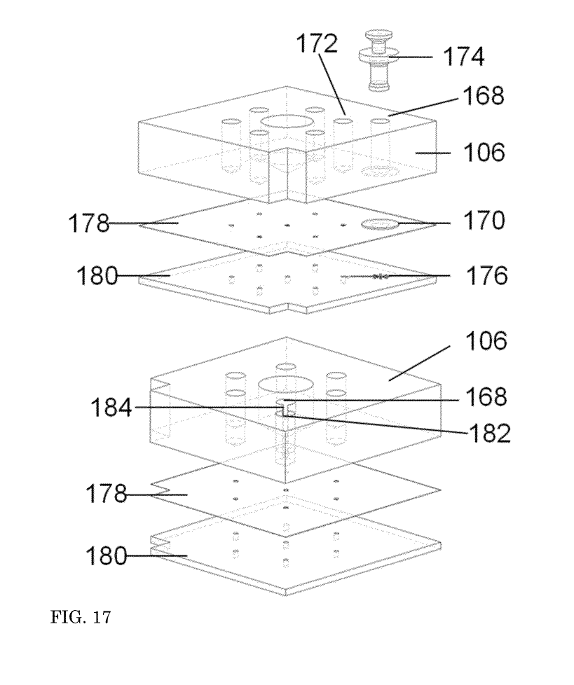

Samples that could use our sample analyzer are typically serum, plasma, urine, and CSF. It is possible to use whole blood as a sample with on-site sample preparation. FIG. 17 shows two examples of onsite plasma preparation with filtration and centrifugation. In Design 1 (FIG. 17 top), a whole blood reception well 168 is introduced on the reagent compartment and multiple layer filters 170 are fixed between whole blood receptacle 168 and the plasma well 172. A plunge-type cap 174 is to seal the whole blood receptacle 168, while pushing blood through filters 170 to a plasma well 172. The plasma well 172 is connected to the fluidic subsystem and the collected solution is used for sample test. The multi-layer filter membrane 170 is sandwiched between two plates. A star channel feature 176 is located at the plasma side of the filter to collect filtered solution, also to supply the support of filter 170. Double-side tapes 178 could be used to form water-proof sealing between layers of membranes and between plate 180 and the membrane. Cell lysis could be controlled with the applied force, which is controlled by the depth of inserted plunge 174 and proper area. The efficiency of plasma collection could reach 25% of whole blood in this design. In Design 2 (FIG. 17, bottom), a cell collection chamber 182 is introduced on the cartridge 106, which is connected to the whole blood receptacle 168 with a narrow gap feature 184. Both wells are located on the line of a centrifugation radius. A cartridge adapter connected to a motor head is used to rotate the whole cartridge 30 and the cell pellets would accumulate in the outside well after centrifugation. Plasma left in the inside well is loaded to the fluidic subsystem after loading the cartridge 30 onto the system through the bottom hole. The efficiency of plasma could reach 50% of the whole blood with proper well designs.

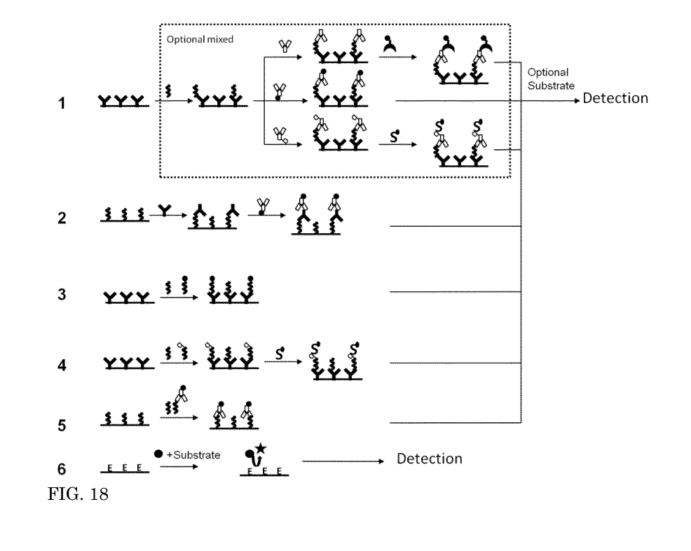

Because of the open modular system design, this invention could easily accommodate various assay methods, as shown in FIG. 18. In theory, any assays that can be captured on site for quantifications could be good candidates, which includes all the sandwich ELISA format with variations (FIG. 18-1), direct ELISA (FIG. 18-2), competitive ELISA (FIGS. 18-3 to -5) and their variations, and direct enzymatic measurements (FIG. 18-6). It is worth noting that it is possible to mix all reaction reagents together and be captured on site with a different binding mechanism, either Ab-Ag or Avidin-Biotin mechanism, which should greatly reduce the assay steps and time involved, thus leading to a much simplified device design.

All the modules used in the fluidic subsystem (pump, valve, sensor, flow cell, etc.) could be combined with quick connects 40a and 40b. It is great for prototype development because of its simplicity to switch different modules. Even for the final version of the device, quick connect-based modular design is a good option for cartridge loading and waste container connection.

An example of complete operation procedures are described below:
1. System preparation, including system validation and priming. A separate priming protocol may be used.
2. Getting the sample(s) and the appropriate assay cartridge(s) 34.
3. Optional sample pretreatment with onsite preparation, according to the protocol.
4. Inserting the assay cartridge 34 into the analyzer through the opening cover, or inserting the reagent 30 and assay 32 compartments separately to the specified locations according to the protocol. Either a drop-in or insert-in mechanisms are used for automatic fluidic connections.
5. Identifying, registering and processing information about the assay cartridge 34 into the sample analyzer by means of a user interface 16.
6. Initiating analysis by inputting a command into a controller 18 located within the analyzer by means of the user interface 16.
7. Monitoring the real time information displayed on the GUI 16 about the status of the assay such as temperature, flow rate, and the potential error messages.
8. Collecting, analyzing, reporting and storing the analytical data by means of the user interface 16.
9. Discarding used cartridges and waste per safety rules and replace with dummy cartridges for idle operations.
10. Troubleshooting according to the on-screen display or the manual.
11. Operating maintenance protocol for normal day-to-day operations and dormant protocol for long term storage.

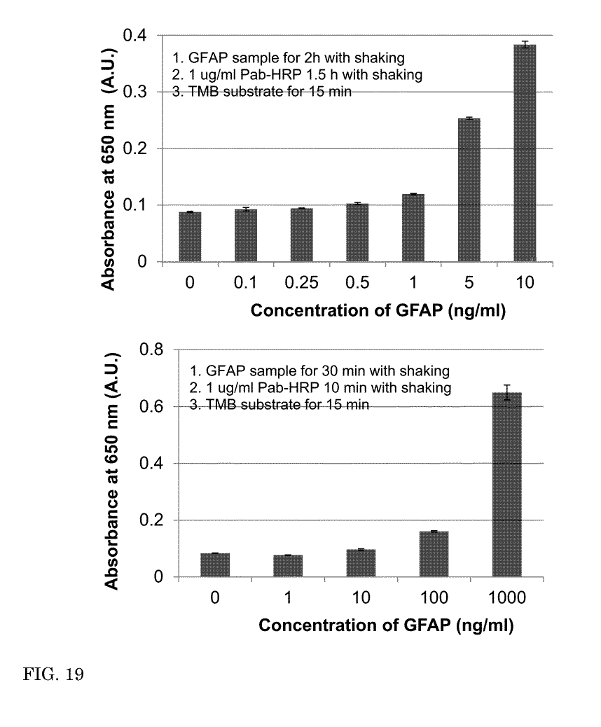

As described before, though 96-well plate assay platform is well accepted as the gold standard for most assays, its performance deteriorates when using an expedited protocol. FIG. 19 shows the decreased performance of 96-well plate assay with accelerated steps according to one test. A standard sandwich ELISA for the detection of GFAP break down product (GFAP BDP) was conducted with total detection time of about 3 hr 45 min. In this test, monoclonal anti-GFAP antibody (Mab) was used as the primary antibody that was immobilized on the ELISA plate. After blocking with blocking buffer, the GFAP sample was delivered into the plate for incubation; then, horseradish peroxidase (HRP) conjugated polyclonal anti-GFAP antibody (Pab-HRP) was introduced for incubation; finally TMB substrate was added for incubation and the HRP enzymatic product was determined by measuring the absorbance on the plate reader. FIG. 19 (top) shows sandwich ELISA for GFAP test with limit of detection (LOD) about 250 pg/ml for 4 hr detection time. Following this, 55 min sandwich ELISA (FIG. 19 bottom) was performed for the detection of GFAP with LOD of about 10 ng/ml. Experimental conditions are shown in the FIG. 19. In this example, GFAP assay with different protocols showed that assays with a 55-minute protocol significantly increases the LOD (by more than an order of magnitude) compared to the normally recommended 4-hour protocol. So expediting the protocol by cutting down the time or number of steps simply deteriorates the sensitivity. As shown later in a comparative example, it is easy for the analyzer of the present invention according to certain implementations to obtain better LOD within 60 minutes.

### Test Example 1

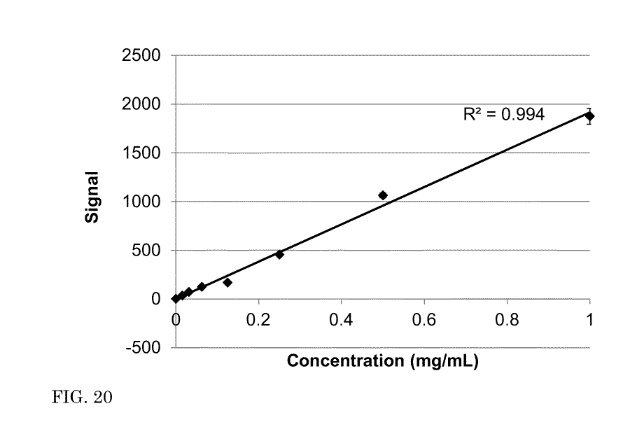

To check the non-assay related system reliability (including flow, detector, quick-connect components, electronics and software), blue dextran solutions with concentration from 0.0156 to 1 mg/mL were injected into a blank cartridge (FIG. 3) and the signals were measured offsite through a microflow cell with absorbance measurement. The test results from ten repeated experiments are shown in Table 3 and graphically in FIG. 20. A linear standard curve is plotted with relative standard deviation (RSD) value below 5% for concentrations over 0.0313 mg/mL dextran, which covers more than 97% of the tested dynamic range zone. For lower concentrations, the reliability is more affected by the capability of the detector and the flow variations. Considering the signals were obtained with moving solutions, the actual variations for assay sites are expected to be even less, which indicates a better detection reliability for on chip detection.

TABLE 3
System reliability test with ten repeated blue
dextran dye test.

| Cone (mg/mL) | Average signal | Stdev | RSD (%) |
|---|---|---|---|
| 0 | 2.49 | 1.72 | 68.9 |
| 0.0156 | 35.55 | 6.54 | 18.4 |
| 0.0313 | 72.18 | 2.03 | 2.8 |
| 0.0625 | 125.13 | 4.80 | 3.8 |
| 0.125 | 168.30 | 5.66 | 3.4 |
| 0.25 | 456.13 | 5.78 | 1.3 |
| 0.5 | 1063.53 | 24.25 | 2.3 |
| 1 | 1875.15 | 81.91 | 4.4 |

### Test Example 2

### Assays with Offsite Detection

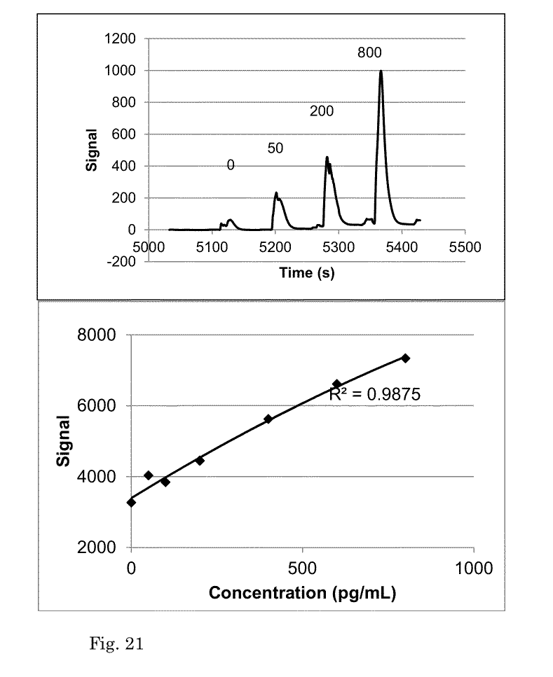

IL6 test with spiked human serum with offsite detection was performed on a test instrument. PMMA capillary tubing coated with mouse anti-IL6 antibody was blocked with blocking buffer and dried for storage. Capillary columns were cut into 10 cm long segments and assembled with the cartridge housing as shown in FIG. 3. Reagent compartments (FIG. 11) were prefilled with standard solutions, washing buffers, secondary antibodies, streptavidin-HRP solution and substrate. After inserting both compartments into the system and loading the sample to the receptacle, the assay was performed with a preconfigured program automatically. A typical offsite real time detection signal with IL6 concentration from 0 to 800 pg/mL is shown in FIG. 21 top. After automatic baseline correction, peak heights at specific timing were measured and the sample concentration could be determined by comparing to the standard solutions. The overall assay time was about 75 min (30 min sample incubation, 15 min secondary antibody incubation, 10 min Streptavidin-HRP incubation and 10 min color development plus washing time), which is much faster than a comparable 96-well ELISA assay (4-6 hours).

Besides the real time calibrators, a predefined master calibration curve could also be combined with an on chip calibrator to further minimize assay variations. As shown in Table 4, the spike recovery test results of IL6 assays at different concentrations were calibrated with a predefined calibration curve (FIG. 21 bottom), which was generated based on three repeated assays. The assay procedure was similar to that described earlier, except the final sample data was calibrated against a real-time calibrator adjusted master standard curve. Except the low concentration range, the spike recovery of IL6 assays are all within 10% variations, which is comparable to traditional 96-well plate detection.

TABLE 4
Spike recovery test of IL6 samples in human sera.

| Spiked conc. (pg/mL) | Measured conc. (pg/mL) | Recovery (%) |
|---|---|---|
| 100 | 68.6 | 69 |
| 200 | 182.4 | 91 |
| 400 | 418.3 | 105 |
| 200 | 184.2 | 92 |
| 600 | 631.6 | 105 |

A panel of IL6 experiments with a total of 15 tests over four days is shown in Table 5. In details, PMMA columns with 500 μm ID were coated with priming antibody and cut into 10 cm lengths. Each reagent cartridge contains five capillary columns. One column is used for sample test and the other four are used for real-time calibration for best measurement accuracy. Three to four tests were performed each day with an 88-min protocol. A system cleaning step was used between assays and fresh cartridges were used for all the tests. The internal standard concentrations are 0, 50, 200 and 800 pg/mL IL6 spiked human sera samples were prepared with human serum with concentration range from 50 pg/mL to 400 pg/mL. The results of the panel of experiments showed that the recovery rates are within 32% of variations, while 14 out of 15 tests are less than 25%. Meanwhile, the precision of the system at different concentrations can also be obtained from this panel of experiments and summarized in Table 5. The overall spike-recovery precision is between 82% to 103% with a less than 20% variation. These results already match most commercial 96-well ELISA platforms with serum/plasma tests, obtained with a smaller footprint, much shorter assay time, and with a fully automated process.

TABLE 5
Panel of IL6 assay tests with spiked
human sera. Total of 15 tests in four days.

| | Spiked | Calculated | |
|---|---|---|---|
| | Concentration | Concentration | |
| | (pg/mL) | (pg/mL) | recovery |
| Test 1 | 100 | 78.85 | 0.79 |
| Test 2 | 100 | 100.31 | 1.00 |
| Test 3 | 100 | 67.97 | 0.68 |
| Test 4 | 217.9 | 183.01 | 0.84 |
| Test 5 | 217.9 | 182.19 | 0.84 |
| Test 6 | 217.9 | 181.49 | 0.83 |
| Test 7 | 217.9 | 212.88 | 0.98 |
| Test 8 | 75 | 64.94 | 0.87 |
| Test 9 | 75 | 56.43 | 0.75 |
| Test 10 | 75 | 63.18 | 0.84 |
| Test 11 | 50 | 57.93 | 1.16 |
| Test 12 | 400 | 451.69 | 1.13 |
| Test 13 | 400 | 498.51 | 1.25 |
| Test 14 | 400 | 374.33 | 0.94 |
| Test 15 | 400 | 321.12 | 0.80 |

### Test Example 3

### Assays with Onsite Detection

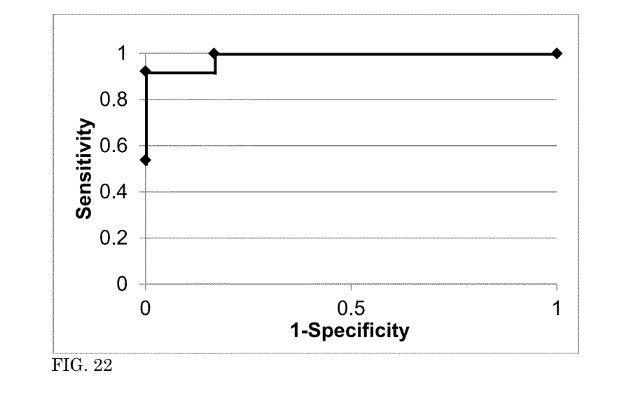

A panel of IL6 test with onsite detection was performed with the example system similar to the one shown in FIG. 1. In details, 1.5 mm thick polystyrene assay chips were manufactured with hot embossing. The chips were batch processed for sealing and antibody coating. The final chips were stored dry in the refrigerator for the panel of experiments. The reagent cartridges were machined in house. All solutions except samples were prefilled in the cartridges and stored in the refrigerator before tests every day. Samples were prepared every day with human sera. After sample loading and cartridge/chip assembly, a 67 min protocol (including priming) was used for all the tests. A washing cycle with dummy chip and washing cartridge was performed between tests. Results of 7 days of 28 sample tests are summarized in Table 6. The results show consistent performance between 6.25 pg/mL and 200 pg/mL. The imprecision is less than 25% for most assay conditions. The variation is higher at 6.25 pg/mL, which is below the claimed 10 pg/mL LOD. The corresponding Receiver Operating Characteristics (ROC) curve with a 30 pg/mL cutoff value is plotted in FIG. 22. It demonstrated 100% sensitivity and 83% specificity.

TABLE 6
Spike recovery test of 28 IL6 sera
with onsite detection.

| Concentration | Measured | | |
|---|---|---|---|
| (pg/mL) | (pg/mL) | stdev | RSD |
| 6.25 | 4.5 | 2.4 | 54.5% |
| 12.5 | 12.1 | 2.1 | 17.7% |
| 25 | 22.1 | 6.1 | 27.7% |
| 37.5 | 37.7 | 9 | 24.0% |
| 100 | 105.9 | 16 | 15.1% |
| 150 | 133.9 | 14 | 10.5% |
| 200 | 174.5 | NA | NA |

Another example of analyte is TBI biomarker GFAP. A panel of GFAP tests in spiked human sera was also performed. In details, 2.2 mm thick white polystyrene chips were manufactured with hot embossing. The chips were sealed and coated with primary antibodies in-house. The final chips were stored dry in the refrigerator for the panel of experiments. The reagent cartridges were machined in-house. All solutions except samples were prefilled in the cartridges and stored in the refrigerator before tests. A modified 67 min protocol (including priming) was used for all the tests. All measurements were finished automatically with integrated detector and control software. A washing cycle with dummy chip and washing cartridge was performed between tests. The intra-assay precision of GFAP test was examined by measuring the same concentration on the same chip at five different concentration levels. As shown in Table 7, the test of GFAP spiked serum samples showed intra-assay CV<15% and the LOD is about 50 pg/mL. Though these results compare well or better to other systems (such as standard 96-well assay (FIG. 19)), we expect them to be further improved with better quality assay chips (e.g. injection molded chips).

TABLE 7
Intra assay CV of spiked GFAP test in human sera.

| Conc. (pg/mL) | Signal (au) | Stdev | CV (%) |
|---|---|---|---|
| 0 | 82719.6 | 9202.8 | 11.1 |
| 50 | 141402.2 | 12093.0 | 8.6 |
| 100 | 227471.0 | 21363.9 | 9.4 |
| 400 | 481911.1 | 59686.8 | 12.4 |
| 800 | 747024.9 | 101731.4 | 13.6 |

The overall GFAP assay performance with onsite detection system was assessed with a series of spike recovery tests. With similar assay setup and protocol, human sera were spiked with certain levels of recombinant GFAP and 15 test results were obtained in straight 4-day tests. The measured values were compared with the expected amount of GFAP spiked (Table 8). This device demonstrated very good recovery (<8% variations) with concentrations above 50 pg/mL spiked samples. The recovery became uncertain when the concentration is below 50 pg/mL LOD.

TABLE 8
Spike recovery test of 15 GFAP sera.

| Spiked Conc. (pg/mL) | Measured Conc. (pg/mL) | Recovery (%) |
|---|---|---|
| 25 | 13.3 | 53.2 |
| 50 | 49.4 | 98.8 |
| 75 | 75.8 | 101.1 |
| 100 | 107.8 | 107.8 |
| 150 | 149.4 | 99.6 |
| 300 | 320.9 | 107.0 |
| 600 | 591.1 | 98.5 |

### Test Example 4

### Adaptation of Competitive Assays

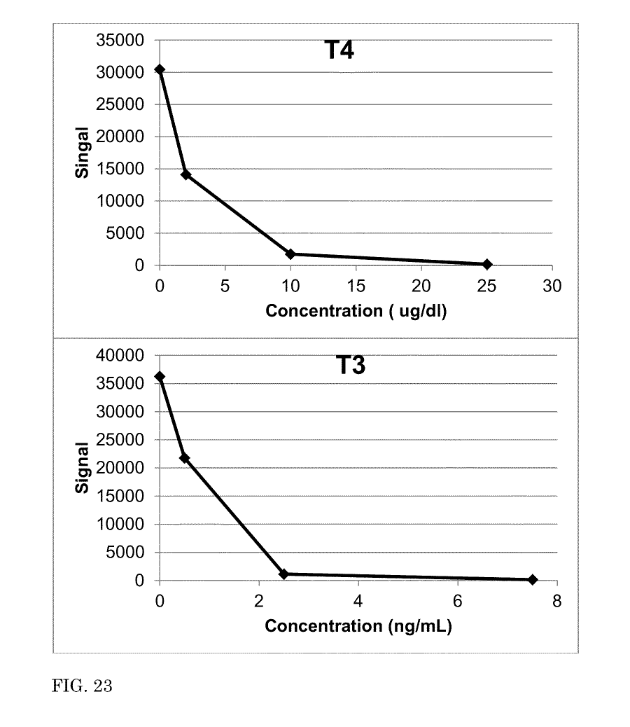

FIG. 23 shows the system performance of competitive immunoassays for T3 and T4 measurements. In details, the assay sites were coated with streptavidin as the capture reagents. Standard/sample solutions were mixed with specific concentrations of HRP labeled T3 (T4) and biotinylated T3 (or T4) antibodies. The mixture was loaded to the assay sites with the device and incubated for 13 min before washing with washing buffer twice. Signals were measured on site after loading substrate through the assay sites immediately. This assay method is actually similar to that in FIG. 18-3 except an additional capturing layer was introduced. The competition happened between sample/standard T3 (T4) and HRP labeled T3 (T4) for the binding of biotinylated antibody, which was captured eventually at the assay site. Washing step removes the unbound enzyme conjugates and the final signal is reversely correlated to the concentrations of sample and standards. The data shown in FIG. 23 is comparable with results from commercial assay kits. It confirms that this invention is capable of measuring analytes with competitive assay methods, which also greatly reduces the assay time required (<20 min).

### Test Example 5

### Simultaneous Detection of Multiple Biomarkers

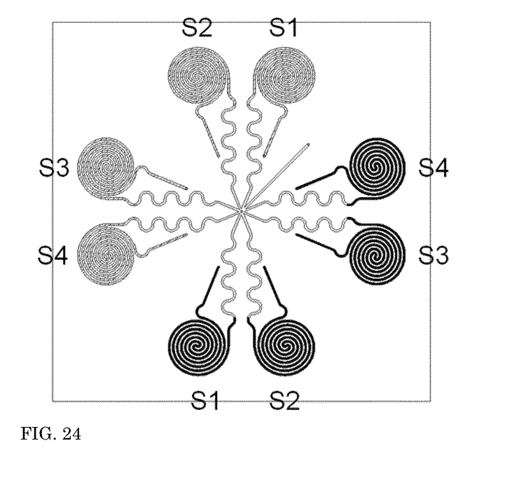

As a platform system, multiple biomarkers have been proved working on the system. Multiple biomarker detection could be achieved with sequential tests by changing assay reagents/chip with one single instrument. However, the total assay time will be multiplied by the number of biomarkers tested. This is not practical unless more instruments are used simultaneously. To solve this dilemma, an eight spiral assay chip was designed and fabricated for simultaneous dual biomarker detection (FIG. 24). An IL6/GFAP dual assay was demonstrated with spiked human sera.

TABLE 9
Sample preparation for dual biomarker test.

| | GFAP (pg/mL) | IL6 (pg/mL) |
|---|---|---|
| Sample 1 (S1) | 0 | 200 |
| Sample 2 (S2) | 50 | 50 |
| Sample 3 (S3) | 200 | 12.5 |
| Sample 4 (S4) | 800 | 0 |

The overall chip dimensions and spiral characteristics remain the same with the eight spiral chips. Two preliminary tests for simultaneous detection of GFAP and IL6 in co-spiked serum samples had been conducted. Four spirals shown in FIG. 24 were used for GFAP (solid black) measurements and the other four spirals were used for IL6 (broken line) measurements. These spirals were carefully coated with their primary antibodies. To minimize the number of reagents, the standard solutions/samples were prepared as mixtures in a reverse concentration order in human sera (cf. Table 9); thus any potential crosstalk of two assay reagents should reveal if present. The secondary antibodies were also a mixture of the two to save number of reagents used. Thus no modification to the reagent cartridge was required. The protocols were modified for eight-spiral chip tests, but the overall assay time was kept the same (67 min including priming time). The total sample requirement is about 100 μL each, which is about doubled compared to single biomarker test. The results are shown in Table 10. Both GFAP and IL6 measurements were comparable to those obtained with four spiral chips on single biomarker measurements. These results clearly demonstrated multi-biomarker simultaneous detection capability of this implementation of the invention.

TABLE 10
Assay results of simultaneous IL6 and
GFAP measurements with eight-spiral chips.

| GFAP | | | |
|---|---|---|---|
| (pg/mL) | Signal | IL6 (pg/mL) | Signal |
| 800 | 8223 | 200 | 16429 |
| 200 | 7469 | 50 | 4034 |
| 50 | 5528 | 12.5 | 2875 |
| 0 | 5141 | 0 | 1987 |

### Test Example 6

### Enhancement with Recursive Sample Loading

Another way to further increase the sensitivity is to use a recursive sample loading strategy. To evaluate this approach, the GFAP assay was performed with all conditions similar to that described earlier, except a modification to the protocol so that the samples would be loaded after all standard solutions. Instead of loading 40 μL sample the same way as the standard solutions at once through the assay spiral, 55 μL samples were loaded four times at one min interval. The overall assay time increased 2 min more to 69 min. The preliminary test result with this approach is summarized in Table 11. Fifteen GFAP spiked human serum samples were tested in 4 days. An enhancement effect from the recursive sample loading was observed compared to previous single loading method. It is lower than the expected value (300% based on four times loading vs one time loading) and lower at low concentration range (average+68% for concentration<150 pg/mL) and higher at high concentration range (average+125% for concentration>150 pg/mL). An adjustment method could be established with more tests to correlate the measured value to the true value, which could further improve the LOD of the system. The potential drawbacks of this approach are longer assay time (because of more sample loading and incubation time) and additional sample volume requirement for extremely low concentration samples. The difficulty is to establish a reliable correlation between the actual and the measured sample concentration after multiple loadings.

TABLE 11
Enhancement effect of recursive
sample loading strategy. 4 days of 15 GFAP
spiked serum samples were tested.

| | Measured | | |
|---|---|---|---|
| Spiked conc | Concentration | | |
| (pg/mL) | (pg/mL) | Ratio | Enhancement |
| 0 | 0 | NA | NA |
| 0 | 0 | NA | NA |
| 25 | 42 | 1.68 | 68.00% |
| 50 | 83 | 1.66 | 66.00% |
| 75 | 117 | 1.56 | 56.00% |
| 75 | 109 | 1.45 | 45.33% |
| 75 | 156 | 2.08 | 108.00% |
| 100 | 181 | 1.81 | 81.00% |
| 150 | 214 | 1.43 | 42.67% |
| 150 | 288 | 1.92 | 92.00% |
| 300 | 730 | 2.43 | 143.33% |
| 300 | 613 | 2.04 | 104.33% |
| 300 | 818 | 2.73 | 172.67% |
| 300 | 727 | 2.42 | 142.33% |
| 600 | 1330 | 2.22 | 121.67% |

The present invention has been described with reference to the foregoing specific implementations. These implementations are intended to be exemplary only, and not limiting to the full scope of the present invention. Many variations and modifications are possible in view of the above teachings. The invention is limited only as set forth in the appended claims. All references cited herein are hereby incorporated by reference to the extent not inconsistent with the disclosure herein. Unless explicitly stated otherwise, flows depicted herein do not require the particular order shown, or sequential order, to achieve desirable results. In addition, other steps may be provided, or steps may be eliminated, from the described flows, and other components may be added to, or removed from, the described systems. Accordingly, other implementations are within the scope of the following claims. Any disclosure of a range is intended to include a disclosure of all ranges within that range and all individual values within that range.

1. A microfluidic sample analysis apparatus, said apparatus comprising:
a. a housing;
b. a multi-layer assay cartridge, comprising:
i. a rigid layer, comprising:
1. at least one microfluidic assay site having a cross-sectional dimension less than 1 mm for receiving a fluid assay;
2. a fluid receptacle; and
3. a microfluidic interface fluidically connected the fluid receptacle and the microfluidic assay site of the rigid layer, and
ii. a barrier layer comprising a barrier material that seals said ridged layer comprising the microfluidic assay site to prevent fluid from leaking out and prevent air from leaking into the microfluidic assay site;
c. a door attached to the housing, wherein the door is sized to receive the assay cartridge within the housing;
d. a rigid chassis located within the housing, wherein the chassis comprises:
i. a platform to receive the multi-layer assay cartridge assembly;
ii. a microfluidic interface to establish fluidic communication with the assay cartridge;
iii. a pump to deliver a fluid into the assay cartridge at a rate of between 1 μL and 1000 μL per minute; and
iv. a detector subsystem, comprising at least one of either an optical detector to detect an optical signal from a sample analyte within a fluid located within the assay cartridge, or an optical source to measure an optical signal from a sample analyte within a fluid located within the assay cartridge or an optical signal from a downstream microfluidic flow cell from the assay cartridge, with a defined path length of at least 1 mm, or an electrochemical-sensing electrode to receive a signal from the fluid located within the assay cartridge and generate an electrical signal in response;
e. an electronic controller in electrical communication with the detector subsystem and the pump to control a rate of flow of the pump and receive and record a reading from the detector subsystem; and
f. a user interface in electrical communication with the electronic controller to provide bi-directional communications with the electronic controller.

2. The apparatus of claim 1, wherein the user interface is a touchscreen display.

3. The apparatus of claim 2, wherein the assay cartridge comprises:
a. a microcapillary structure;
b. a first magnetic quick-connect in fluidic communication with the microcapillary structure; and
wherein the platform comprises a second magnetic quick-connect matched to the first magnetic quick-connect for allowing fluidic communication to the microcapillary structure.

4. The apparatus of claim 3, wherein the microcapillary structure comprises external microcapillary tubing inserted through a through-bored hole within the assay cartridge.

5. The apparatus of claim 1, wherein the rigid layer comprises a spiral configuration to create a serpentine microfluidic channel comprising a cross-sectional microchannel dimension and channel-to-channel spacing of less than 1 mm.

6. The apparatus of claim 1, wherein the rigid layer comprises a plurality of spiral configurations to create a plurality of serpentine microfluidic channels, and wherein an area of the plurality of serpentine microfluidic channels is less than 1.5 in×1.5 in.

7. The apparatus of claim 6, wherein each of the plurality of serpentine microfluidic channels are connected to a common fluidic port.

8. The apparatus of claim 7, wherein the rigid layer further comprises one or both of elongated or site restricted features to preclude liquid intermixing between the plurality of serpentine microfluidic channels.

9. The apparatus of claim 8, wherein the rigid layer further comprises a bypassing microchannel with reagent exchange and reagent removal.

10. The apparatus of claim 1, further comprising a sealing layer attached to the rigid layer, wherein the sealing layer is comprises of at least one of optically clear or translucent colored materials to allow for optical detection within the assay cartridge.

11. The apparatus of claim 10, further comprising an interconnect layer in communication with an electrochemical sensor.

12. The apparatus of claim 6, wherein at least one of the plurality of spiral configurations is configured as an internal calibration site to receive a calibration solution.

13. The apparatus of claim 1, wherein the assay cartridge further comprises a reagent compartment comprising a side covered by a rigid lid, wherein the rigid lid comprises two sides, at least one micro-aperture, and a quick-connect embedded structure opposite the micro-aperture, with both sides of the rigid lid sealed with barrier membranes for long-term storage of a reagent in the reagent compartment.

14. The apparatus of claim 1, wherein the assay cartridge further comprises a reagent compartment, wherein the reagent compartment comprises:
a. a plurality of fluid receptacles, wherein at least a first fluid receptacle of the plurality of fluid receptacles comprises a first fluid receptacle side covered by a rigid lid comprising at least one first fluid receptacle micro-aperture, and at least a second fluid receptacle of the plurality of fluid receptacles comprising a second fluid receptacle side covered by an elastomeric membrane, a rigid lid with at least one second fluid receptacle micro-aperture, and an adhesive membrane that seals the second fluid receptacle micro-aperture, wherein the platform comprises at least one microfluidic line and a rigid hollow micro-needle that establishes fluidic contact between the second fluid receptacle by piercing through the elastomeric membrane and the fluidic line located on the other side of the platform; and
b. a spring-loaded guard plate to prevent accidental exposure to the micro-needle during insertion by a user.

15. The apparatus of claim 1, wherein the assay cartridge comprises an integrated chip assembly comprising:
a. an assay layer comprising a rigid solid material, the assay layer comprising a plurality of microfluidic assay sites; and
b. a reagent compartment, wherein the assay layer is secured against the reagent compartment to form a rigid lid on one side of the reagent compartment, with a set of assay layer fluidic ports connected through a matching set of reagent compartment fluidic ports to provide access between the reagent compartment and the plurality of microfluidic assay sites.

16. The apparatus of claim 1, wherein the assay cartridge comprises:
a. a plate comprising at least one reagent compartment and at least one assay site, wherein the reagent compartment and the assay site are not fluidically connected on the plate;
b. a replaceable septum fittable over the plate to prevent ingress or ingress to the at least one reagent compartment and the at least one assay site; and
c. a replaceable connection channel chip that may be fitted onto the plate in place of the septum, the connection channel chip comprising at least one channel fluidically connecting the at least one reagent compartment and the at least one assay site.

17. The apparatus of claim 1, further comprising a manifold or multichannel valve fluidically connected to the pump and a plurality of microfluidic connections.

18. The apparatus of claim 1, wherein the detector subsystem comprises a wide-aperture camera covering within a field of view of the wide-aperture camera a plurality of assay sites on the assay assembly.

19. The apparatus of claim 1, wherein the detector subsystem comprises a wide-aperture photomultiplier and an opaque mechanized shutter with at least one defined aperture, and wherein the controller is configured to expose at one time a site within the assay assembly by means of controlled mechanical movement of the opaque mechanized shutter.

20. The apparatus of claim 1, wherein the detector subsystem lacks an external light source and receives an optical signal from a luminescent species within the assay assembly.

21. The apparatus of claim 1, wherein the detector subsystem receives at least one of a colored light or fluorescent signal from a species within the assay assembly.

22. The apparatus of claim 1, wherein the controller records and analyzes a rate of signal development and an end-point signal from the assay assembly.

23. The apparatus of claim 1, further comprising a chip adapter base within the chassis to establish a leak-proof seal with the fluidic connection with the assay cartridge comprising at least one O-ring or elastomeric membrane gaskets.

24. The apparatus of claim 1, wherein the assay cartridge is an assay chip, and wherein the apparatus further comprises:
a. a linear actuator;
b. a spring-loaded actuator connected to the linear actuator; and
c. a pressure sensor positioned adjacent the assay chip and in electrical communication with the electronic controller,
wherein the linear actuator applies a desired pressure, as measured by the pressure sensor, at the assay chip in order to seal the microfluidic interface.

25. The apparatus of claim 1, wherein the assay cartridge further comprises an embedded filtration system wherein a source of external pressure drives a fluid through the filtration system into the assay cartridge.

26. The apparatus of claim 1, further comprising a loading receptacle in fluid communication with the assay cartridge, wherein the loading receptacle comprises a centrifuge to separate a fluid from residue wherein the residue is not directed into the assay cartridge.

27. The apparatus of claim 1, wherein the assay cartridge is prefunctionalized with a chosen reagent to trap a specific analyte present in a fluid passing through the assay cartridge.

28. The apparatus of claim 1, wherein the assay cartridge is prefunctionalized with a plurality of chosen reagents to trap a plurality of specific analytes present in a fluid passing through the assay cartridge.

29. The apparatus of claim 1, wherein the assay cartridge comprises a glass or plastic selected from the group consisting of polystyrene, polycarbonate, poly(methyl methacrylate), polyester, and polymers and copolymers of olefins and cyclic olefins.

30. The apparatus of claim 1, further comprising a temperature regulating system within the housing and in electrical communication with the controller, wherein the temperature regulating system comprises:
a. a temperature sensor; and
b. a temperature source capable of at least one of heating or cooling the assay cartridge in response to a command from the controller

31. The apparatus of claim 1, further comprising:
a. a pressure sensor within the housing and in electrical communication with the controller; and
b. a manifold in communication with the pressure sensor and in fluidic communication with a plurality of microfluidic channels within the assay cartridge.

32. The apparatus of claim 1, further comprising a flow sensor in fluidic communication with at least one microfluidic channel within the assay cartridge.

33. A method of analyzing a microfluidic sample utilizing the apparatus of claim 13, the method comprising the steps of:
a. inserting the multi-layer assay cartridge assembly through the door attached to the housing;
b. inserting a reagent compartment into the reagent compartment of the assay cartridge;
c. receiving at the user interface identifying information about a reagent in the reagent compartment;
d. receiving at the user interface a command to begin analysis of a fluid;
e. at the electronic controller, collect and analyze data concerning an analyte in the fluid; and
f. display at the user interface data concerning an analyte in the fluid.
